(19) United States
(12) Patent Application Publication
(10) Pub. No.: US 2013/0041145 A1
(43) Pub. Date: Feb. 14, 2013

(54) METHOD FOR ISOLATING RNA FROM WHOLE BLOOD SAMPLES

(71) Applicants: Christoph KIRSCH, Pulheim (DE); Claudia Beyard, Zuelpich (DE); Markus Meusel, Wuerselen (DE); Thomas Zinn, Dueren (DE); Carolin Wagner, Dueren (DE); Klaus Moeller, Eschweiler (DE)

(72) Inventors: Christoph KIRSCH, Pulheim (DE); Claudia Beyard, Zuelpich (DE); Markus Meusel, Wuerselen (DE); Thomas Zinn, Dueren (DE); Carolin Wagner, Dueren (DE); Klaus Moeller, Eschweiler (DE)

(21) Appl. No.: 13/572,072

(22) Filed: Aug. 10, 2012

(30) Foreign Application Priority Data
Aug. 11, 2011 (DE) 10 2011 080 853.1

(51) Int. Cl. C07H 1/08; C12N 9/50

(52) U.S. Cl. 536/25.41; 435/219

(57) ABSTRACT

The present invention relates to a method for isolating RNA from a whole blood sample, comprising the steps bringing the sample into contact with an aqueous lysis solution containing at least one lysis substance in a concentration of 1.5 mol/1 to 7 mol/1 and at least one detergent, simultaneously or subsequently adding a proteinase, in particular proteinase K, then incubating the solution for at least partial enzymatic digestion and lysis of the sample so that a lysate is obtained, and isolating the RNA from the lysate.

### CROSS-REFERENCE TO RELATED APPLICATIONS AND CLAIM TO PRIORITY

This application is related to application number 10 2011 080 853.1, filed Aug. 11, 2011 in the Federal Republic of Germany, the disclosure of which is incorporated by reference and to which priority is claimed.

### FIELD OF INVENTION

The present invention relates to a method for isolating RNA from a whole blood sample.

### BACKGROUND OF THE INVENTION

Blood is an accessible source for animal or human RNA. Blood is made up of cellular components and plasma, the cellular percentage varying according to sex and age, but generally being on average 44%. Due to this high cellular percentage, blood is often also called “liquid tissue”.

Despite this high cellular percentage in blood, the number of metabolically active cells is extremely small overall. Thus the DNA- and RNA-carrying leucocytes only make up approximately 0.1 to 0.2% of the cellular fraction, the largest cell population by far being formed by mature erythrocytes. These have no nucleus, however, i.e. they do not generally carry any RNA, but instead all the more haemoglobin for conveying oxygen in the blood.

Due to the greater presence of DNA in the blood in relation to RNA, the isolation of genomic DNA from blood is relatively easy to bring about. The high stability of the DNA is also advantageous here. In contrast, the isolation of intact RNA is extremely challenging, particularly when the RNA is to be isolated from the blood sample as completely as possible. As already indicated above, this is due on the one hand to the small percentage of RNA-carrying cells.

The high concentration of RNases in the blood, which are present intra-cellularly and extra-cellularly, constitutes a further difficulty factor. If the RNases are not inactivated as quickly and completely as possible, these lead to degradation of the RNA, and so to a further reduction of the RNA concentration.

A further problem is the required separation of the haemoglobin because the iron-carrying haem is an extremely potent inhibitor of the polymerase chain reaction (PCR).

Irrespective of the aforementioned biochemical difficulties which arise when extracting RNA from blood samples, the very high viscosity of blood is sometimes problematic. Especially with the high cellular percentage (haematocrit), which can occur for example due to illness, whole blood is particularly viscous and therefore difficult to process.

Most of the methods used nowadays in order to isolate RNA from whole blood focus on the separation of the leucocytes containing RNA from the rest of the blood with its cellular and cell-free components.

The original method for obtaining a pure leucocyte fraction is density gradient centrifugation (Böyum, 1968; Isolation of mononuclear cells and granulocytes from human blood. Scand. J. Clin. Lab. Invest. 21 (Suppl. 97): 77-89). An alternative is offered by so-called CPT Tubes (Becton, Dickinson and Company, BD Vacutainer CPT Cell Preparation Tube) which contain a polymer gel and allow the leucocyte fraction to be removed after adding the blood and centrifugation. However, these complex methods are not suitable, or are only suitable to a limited extent, for the isolation and analysis of RNA because the cellular RNA profile is not stabilised or “frozen”. Over the course of the processing changes inevitably occur in the gene expression, and so analysis of the expression patterns is made difficult or impossible. In order to analyse the RNA of the samples it is therefore necessary to control the factors that influence the gene expression very stringently. If—even for just a short time—non-physiological conditions are created (e.g. by centrifugation, cell collection, dilution, etc.), an adverse effect upon the gene expression can not be ruled out.

One well-established method for enriching the leucocytes is selective erythrocyte lysis (Karavitaki et al., 2005, Molecular staging using qualitative RT-PCR analysis detecting thyreoglubulin mRNA in the peripheral blood of patients with differentiated thyroid cancer after therapy. Anticancer Research 25: 3135-3142). This method is, furthermore, described in EP 0 875 271 and U.S. Pat. No. 5,702,884. In simple terms, erythrocytes are lysed by adding several volumes of ammonium chloride solution or other selectively acting formulations, while the leucocytes remain intact under the chosen conditions. After centrifugation the leucocyte pellet is washed, resuspended, and the RNA is isolated from the cells. One great disadvantage is the unwieldy increase of the specimen volume, associated with further dilution of the specimen because hypotonic conditions must generally be set by adding a number of volumes of the lysis solution. The repeated centrifugation steps are also time-consuming and can not be automated, and this is a serious disadvantage. Due to the non-physiological conditions during dilution, a change to the gene expression profiles can furthermore occur which makes subsequent analysis difficult. The large amount of time required by this method promotes this effect even further. Since during the selective lysis the RNases are not suppressed any further, the quality and quantity of the isolated RNA is generally low.

Another possibility for separating leucocytes from other blood components is the isolation of a so-called buffy coat. Here, anti-coagulated blood is centrifuged with low acceleration (approx. 2,000×g). The buffy coat with predominantly white blood cells can be removed as a layer between the plasma and the packed erythrocytes. It is advantageous here that the leucocytes are collected under almost physiological conditions, and dilutions are totally lacking here. However, the processing is time- and work-intensive, requires a high degree of practical experience and skill, and can not rule out changes to the gene expression patterns by centrifugation and processing. There is also the risk of contact with potentially infectious sample material, as well as the impossibility of automating this process.

A further possibility for enriching leucocytes is described by US 2005/0208501 A1. Here a membrane which selectively binds leucocytes from whole blood is used. Originally such membranes were used in blood transfusions in order to rule out graft vs. host illnesses in those receiving the blood. For the extraction of RNA from whole blood the latter is conveyed (centrifuged) via the membrane. The leucocytes bind and can then by lysed. In comparison with density gradient centrifugation or buffy coat extraction, this method is distinctly simpler, nevertheless a change to the gene expression profiles can not be ruled out here either because the samples are centrifuged “non-physiologically” and cells are bound to a membrane.

Disadvantages of all of the methods which focus on the separation of leucocytes and the remaining components of the blood are the time- and work-intensive processing, associated with high costs. In some methods there is also the fact that potentially dangerous pathogens in the blood (e.g. viruses) are not inactivated and remain potentially infectious for the user. Likewise, the change in the gene expression profiles is undesired and can not be totally ruled out. Here, rapid, immediate lysis, which inactivates RNases efficiently and so rules out any change to the RNA pattern, would be helpful. Finally, the impossibility of automation must be emphasised because, for example, centrifugation steps can not be implemented on well established laboratory machines, or only with considerable expense.

As an alternative to the previous separation and isolation of leucocytes, a whole blood specimen can also be lysed directly. Often lysis buffers with guanidinium thiocyanate (GuSCN) are used for the lysis of whole blood and the isolation of RNA (Tan and Yiap, “DNA, RNA and Protein Extraction: The Past and The Present”, Journal of Biomedicine and Biotechnlogy, Vol. 2009, Article ID 574398). The chaotropic salt leads to direct lysis of the cells, and at the same time RNases are directly inactivated. GuSCN also leads to the inactivation of pathogens which are present as potential contaminations in the blood, and this increases user safety.

EP 0 818 542 A1 describes, for example, blood lysis brought about by GuSCN used in powder form. The latter is then brought into a solution by the contact with blood. By means of the solution displaced over time and the low concentration in the blood sample at the start, it can not be ruled out that part of the liberated RNA is already degraded before the effective active concentration of the GuSCN finally brought into a solution is sufficiently high.

In WO 00/09746 a vessel for the extraction of blood is described in which an aqueous solution comprising guanidinium salt, a buffer substance, a reduction agent and/or a detergent is present. If blood is poured into this vessel, the cells are lysed and the RNA is stabilised. After storing the blood samples the RNA can be extracted by well established methods for the isolation of nucleic acid.

Other methods are based on the use of cationic detergents (Macfarlane, U.S. Pat. No. 5,010,183 and U.S. Pat. No. 5,728,822) or lithium chloride/urea.

Commercial products which use direct lysis in order to isolate RNA from whole blood include, for example, the PAXgene Tubes made by PreAnalytix (WO 02/056030). Here the lysis of the blood is implemented with the aforementioned cationic detergents. These tubes are the most suitable for the stabilisation of RNA in blood samples. The RNAs are thus protected, and the expression profiles remain unchanged, even when stored for a number of days at room temperature (for example when sent by post). If the nucleic acids (RNA) are then to be isolated from the tubes after storage, pelletizing initially takes place by centrifugation. The pellet, which contains the insoluble nucleic acids and proteins, is then washed, resuspended, and then the RNA is isolated by classical methods (for example by binding to silica membranes under high salt conditions).

Other commercial tubes for the stabilisation of RNA are the Tempus Blood RNA tubes made by Applied Biosystems. Here guanidine hydrochloride (GuHC1), MOPS and NaCl containing solutions are used.

Apart from for the stabilisation of RNA, direct lysis is only used in a few cases. In the RiboPure kit (Ambion, LifeTechnologies) a whole blood sample is lysed with the aid of phenol/chloroform. In this method the sample is treated according to Chomczymski and Sacchi (1987, Single step method of RNA isolation by acid guanidinium thiocyanate-phenol-chloroform extraction. Anal. Biochem, 162(1)), and with a GuSCN/phenol/chloroform mixture with a low pH value. Under the acid conditions the RNA remains in the aqueous upper phase, whereas DNA and denatured proteins remain in the interphase and the lower organic phase. Generally the RNA is then desalinated and cleansed by precipitation with isopropanol. Even if some of the disadvantages from the prior art are avoided with this approach (e.g. immediate and complete lysis), one absolutely essential disadvantage of this very complex method is the toxicity of phenol, and so from the point of view of the user, these methods should be dispensed with as far as possible. Furthermore, the processing of the 2-phase system can practically not be automated, and so the throughput remains low.

A further method is based upon the “Total RNA” kit (Applied Biosystems) for the ABI Prism 6100 Nucleic Acid Prepstation (WO 2005/003346 A1). Whole blood samples are diluted in a first step at a ratio of 1:1 with PBS (phosphate buffered saline). These samples pre-processed in this way are then lysed with a lysis buffer containing GuHCl and the RNA is bound to a membrane in a vacuum. The dilution with PBS leads on the one hand to an unwieldy increase in volume and is associated with the risk, as are all of the methods in which the blood sample is conditioned and changed before the lysis, that the gene expression is changed due to the non-physiological conditions. The dilution supposedly results from the necessity of also controlling the viscosity of the sample. Due to the processing in a vacuum there is also the risk that the membrane will become clogged up.

The direct lysis of blood is also described in U.S. Pat. No. 7,927,798. Here the lysis must be implemented such that inhibition of the RNA amplification is prevented. Differently from the conventional PCR-based method, the RNA is amplified by means of the so-called bDNA (branched DNA) technology. Here the RNA is immobilised by means of probes on a solid phase and amplified by repetitive, sequential hydridisation with further probes. Detection finally takes place by means of chemiluminescence. Due to the direct evidence without purification, the quantity of blood used is limited. This fluctuates between 1 and 30 μL with a final volume of 150 μL. The sample is therefore diluted by a factor of 5-150.

Due to the following hybridisation of the RNA one can not use high-molar lysis solutions or other formulations which disrupt binding and hybridisation. Further disadvantages here are the limitation of the sample material because only the smallest of sample quantities are used, the associated low sensitivity, and the risk of inhibiting the subsequent analysis because without the normal purification of the nucleic acids it can not be guaranteed that any inhibitors have been removed.

### SUMMARY OF THE INVENTION

The object underlying the present invention is to make available a method for isolating RNA from whole blood samples that enables the direct processing of samples and can in particular be implemented without previously stabilising the nucleic acids, and the most complete possible isolation of the RNA contained in the sample material is thereby made possible. In addition, the method should offer the possibility of automated sample processing.

This object is achieved by a method for isolating RNA from a whole blood sample, comprising the steps

a) bringing the sample into contact with an aqueous lysis solution containing at least one lysis substance at a concentration of 1.5 mol/L to 7 mol/L and at least one detergent,

b) simultaneous or subsequent addition of a proteinase, in particular proteinase K,

c) then incubating the solution obtained according to step b) for at least partial enzymatic digestion and lysis of the sample, a lysate being obtained, and

d) isolation of the RNA from the lysate.

Surprisingly, it has been shown that by the combined use of a lysis substance in the specified concentration ranges and the simultaneous or subsequent addition of a proteinase, total lysis of whole blood samples can be achieved, the concentrations of lysis substance according to the invention not essentially affecting the activity of the proteinase, and so high viscous whole blood samples can also be processed automatically.

At the same time the lysis substance concentration is sufficiently high in order to inactivate the RNases present in the sample in the shortest time. Furthermore, the lysate obtained by means of the method according to the invention enables binding of the RNA to a solid phase under the usual conditions, such as for example by adding ethanol.

By means of the limited use of liquid chemicals according to the invention the overall volume of the sample is not increased, moreover, when processing the sample to the extent as is partially the case in the methods known from the prior art, which often even provide dilution of the sample before the latter is actually processed. In this way the method according to the invention can be implemented in relatively small reaction vessels, and this considerably simplifies the automated sample processing.

In this case the isolation of RNA is understood as meaning that the RNA is liberated as far as possible from the other sample components, in particular from proteins, cell fragments and the like. The removal of further nucleic acids such as DNA is optional, however, and is dependent upon whether the latter are disruptive in the further processing or analysis of the RNA.

In general the method according to the invention is simple to implement and, as already discussed above, can easily be automated. The method according to the invention thus enables whole blood lysis, omitting all of the centrifugation steps as partially provided in methods known until now. Furthermore, the method according to the invention does not require previous pre-treatment or conditioning of the samples, and this not only reduces the processing complexity, but also reduces the risk of possible contamination. Therefore, the specimen is preferably not subjected to any previous processing step before bringing into contact with the aqueous lysis solution.

A further advantage of the present invention is that the method can be implemented without the use of toxic formulations such as phenol/chloroform. Moreover, the method according to the invention is comparably insensitive to fluctuations in the haematocrit, and so the RNA extraction can be processed by normal methods using membranes suitable for RNA isolation without the risk of clogging. Furthermore, the method according to the invention enables the extraction of RNA from whole blood with high quality and yield.

The RNA that can be isolated with the aid of the method according to the invention comprises in principle all of the RNA occurring in the whole blood, such as for example mRNA, rRNA and miRNA, to name just a few.

Proteinase K, or other proteinases with a broad substrate spectrum (preferably with endo- and/or exopeptidase activity) and mixtures of the latter, for example, can be used according to the invention as proteinases, proteinase K being particularly preferred because in this way the viscosity of the whole blood sample can be reduced particularly efficiently. The quantities of proteinase added can be, for example 0.1-5 mg/ml whole blood, preferably 0.5 to 1 mg/ml.

According to one preferred embodiment of the method according to the invention, RNases contained in the solution in step c) are totally inactivated as far as possible. This is brought about with the aid of the concentrations of the lysis substance provided according to the invention.

The integrity and stability of the isolated RNA can be used as a measure of the inactivation of RNases. Only if RNases are inactivated efficiently and as far as possible during the lysis and the sample decomposition, the isolated RNA shows distinct rRNA bands of a known size during analysis by means of denaturing gel electrophoresis or by means of RNA assay on the Agilent bioanalyzer. In human blood samples the RNA (18 and 29S rRNA) appears as two distinct bands, of which the 28S rRNA is clearly more pronounced. Since RNA is very sensitive to the ubiquitous RNases, it is recommended that isolated RNA be frozen or at least be stored at 4° C. If isolated RNA (to be seen on clear 18 and 29S rRNA bands), stored e.g. for one day at room temperature, is analysed once again by means of RNA gel or bioanalyzer, with the presence of the two distinct bands one can conclude that there is as far as possible a total absence of RNases. If the isolated RNA were contaminated with RNases, the RNA would be broken down by incubation at room temperature; the differences would be obvious on the gel.

In another preferred way, the aqueous lysis solution contains the lysis substance at a concentration of 2 mol/1 to 6 mo/l, in particular of 2.5 mol/l to 5 mol/l, preferably of 3 mol/l to 4 mol/l. These concentrations are particularly advantageous because the latter are on the one hand sufficiently high in order to inactivate the existing RNases efficiently and totally before the latter break down the existing RNA. On the other hand, these concentrations are chosen such that there is no stronger inhibition of the proteinase, and so the sample viscosity is reduced to a sufficient extent during the incubation so that the possibility of automated sample processing remains.

The whole blood presented for the method according to the invention can derive from human origin or from other species, such as for example animals. Moreover, the blood can be taken freshly or be in frozen form and derive from commercially available removal systems with normal anticoagulants (EDTA, citrate, heparin).

In another preferred way, the aqueous lysis solution is mixed with the sample at a volume ratio of 1.8:1 to 1:1.8, in particular of 1.5:1 to 1:1.5, preferably of 1.2: to 1:1.2. The aforementioned mixture ratio is particularly preferably approximately 1:1. The aforementioned mixture ratios are particularly advantageous because in this way an unnecessary increase in the sample volume can be avoided by the solutions used.

According to a further advantageous configuration of the method according to the invention, in step b) the proteinase is added as a solid together with a solvent or as a proteinase solution, the respective quantity of liquid being set such that the quantity of liquid is at most 20% of the volume comprising the sample and the aqueous lysis solution, in particular at most 10%. By means of the aforementioned measure unnecessary dilution of the sample and an increase of the sample volume is also avoided, and this has an advantageous effect upon the possibility of automating the method according to the invention.

In another preferred way the sample can be brought directly in contact with the lysis solution in step a), there being no further addition of a further solvent until the completion of step c), and so no further dilution occurring, no stabiliser being added and/or not being centrifuged. With the aid of these measures the complexity of the method according to the invention can be limited, and this reduces its susceptibility to errors and further improves the robustness of the method and the possibility of automation.

In this connection it is particularly advantageous if the overall volume comprising the sample, the aqueous lysis solution and the proteinase exceeds the initial volume of the sample by no more than the factor 2.5, preferably by no more than the factor 1.5, by the completion of step c).

A series of compounds are considered for the lysis substances that can be used within the framework of the method according to the invention. Within the context of the present invention, a lysis substance is understood here as a compound which is capable of liberating biomolecules from the whole blood sample, and at the very least the RNA. The lysis substance can therefore contain a chaotropic salt or mixtures of different chaotropic salts. Here the chaotropic salts are selected in particular from thiocyanates such as sodium thiocyanate, potassium thiocyanate and guadinium thiocyanate, urea, perchlorate salts such as sodium perchlorate and/or potassium iodide or mixtures of the latter. Of the aforementioned compounds thiocyanates are preferred because this compound is capable in a particular way of deactivating RNases, guadinium thiocyanate being particularly preferred.

According to one particularly preferred embodiment of the method according to the invention the lysis solution contains magnesium ions, in particular at a concentration of 10 to 1000 mmol/l, preferably of 100 to 600 mmol/l, more preferably 150 to 400 mmol/l. The addition of magnesium ions is particularly advantageous because in this way the RNA yields can be increased.

In a way that is also preferred, provision can be made such that the lysis substance includes potassium and/or sodium thiocyanate and the lysis solution contains calcium ions, the calcium ion concentration being in particular 10 to 150 mmol/1, preferably 50 to 100 mmol/l. By this combination too the RNA yield of the method according to the invention can be increased, simultaneous use with magnesium ions as described above also being possible.

Any water soluble salts of the aforementioned metals can in principle be used as a source for magnesium ions and calcium ions provided the cations of these salts are chemically and biologically inert under the conditions of the method. This applies, for example, to the chlorides of magnesium and calcium.

In a way that is also preferred, the lysis solution is free from further bivalent or trivalent metal ions, i.e. apart from magnesium and calcium ions. This is particularly advantageous because it has been shown that magnesium and calcium ions have an advantageous effect upon the RNA yield, but other multivalent metal ions generally lead to the precipitation of specimen components. This results in a considerable reduction of the RNA yield and moreover makes automated sample preparation difficult because, for example, membranes used for the isolation are clogged up by the precipitated components.

Provision is made according to the invention such that the lysis solution contains a detergent. In principle, any detergents that can be used for biochemical extraction processes for cell nucleus components can in principle be used here, non-ionic detergents and mixtures of the latter being particularly preferred. The detergent supports the lysis, releases lipids of the lysed blood cells and in this way prevents clogging of membranes used for the separation. The use of polyoxyethylene-20-cetyl ether (Brij 58®), N-lauroyl-sarcosine and mixtures of the latter has a particularly advantageous effect upon the quality of the extracted RNA and the yield of the latter. Other detergents, such as for example Tween20 can be used on their own or in mixtures with the aforementioned detergents.

The quantities of detergents used can be varied over wide ranges. The lysis solution can thus, for example, contain an overall detergent content of 10 to 200 g/l, preferably from 30 to 100 g/l.

The lysis solution used according to the invention can contain further components in addition to the aforementioned ingredients which are chosen in particular from buffer substances, enzymes, reduction agents and/or alcohols.

The isolation of RNA from the lysate in step d) can in principle be undertaken in any way known in its own right. Advantageously the RNA is attached to a solid phase for isolation from the lysate, desirably followed by one or more washing steps.

Solid phases are understood to mean water-insoluble materials to which nucleic acids bind in the aqueous phase in the presence of high ionic strength. These are for example porous or non-porous mineral particles such as silica, glass, quartz, zeolites or mixtures of the latter. The aforementioned particles can, moreover, be in the form of magnetic particles. When using the aforementioned magnetically modified particles, which are also known as magnetic beads, one can revert to magnetic separation. Such procedures are known in principle to the person skilled in the art. Moreover, when using non-magnetic particles, separation can be undertaken by sedimentation or centrifugation.

The solid phase can be in the form of a sedimenting powder or a suspension, or however in the form of a membrane, a filter layer, a frit, a monolith or some other solid body. Of the aforementioned possible embodiments silica membranes or glass fibre fleeces are particularly preferred for the method according to the invention. Conveyance of the pre-treated solution by these materials can be implemented purely by gravitation or by centrifugation, or also by applying a negative pressure.

With particularly preferred representatives, the aforementioned solid phase in the form of a membrane or a fleece is fixed in a hollow body with an inlet and outlet opening with one or more layers. Such hollow bodies are known to the person skilled in the art, for example as a Minispin centrifuge body. The binding, washing, separation and elution steps can thus be conveyed by centrifugal force or a vacuum in the Minispin centrifuge bodies or Minispin columns.

In another preferred way the attachment of the RNA to the solid phase can be supported by a binding agent being added to the lysate. An alcohol, for example, preferably ethanol and/or isopropanol, and a corresponding alcohol/water mixture, such as an ethanol/water mixture can be used for this purpose. The ethanol content can be in particular between 50 and 95 vol. %, for example 70 vol. % ethanol.

Within the framework of the method according to the invention provision can, moreover, be made such that the DNA is at least partially, preferably almost totally removed, in particular by DNase digestion. Whether the DNA is removed or not depends on whether the latter is disruptive to the subsequent analysis. The DNA can be removed at any time after the sample lysis has been carried out. It is advantageous, for example, to break down the DNA enzymatically after attaching the RNA to a solid carrier, i.e. to subject it to DNase digestion. This is because typically, under the binding conditions of the RNA, the DNA also binds, at least partially, to the solid carrier on the surface of which the DNA can then be selectively broken down.

The DNase digestion can take place during the purification process, e.g. after binding the nucleic acids to a solid phase (so-called “on-column” digestion), or however after isolation of the RNA in solution. The DNA is generally detected by means of PCR. Total removal of the DNA, such that there is no PCR amplification either by means of by short fragments of multicopy targets, is technically almost impossible, purely due to the fact that there are specific DNA sequences and species which can not be degraded by means of DNase digestion. Nevertheless, the removal of DNA by means of DNase digestion is significant: The DNase digestion during the isolation of RNA from whole blood samples thus generally leads to a shift of the cp values in the real time PCR by 8-10 cycles. Due to the semi-logarithmic connection between the cp value and the DNA concentration this means for example enrichment by the factor 250-1000.

Procedures known in their own right can be used to extract the isolated RNA. The isolated RNA can thus, for example, be eluted or desorbed from the solid phase. For this purpose an elution solution can be used, for example RNase-free water or only slightly buffered aqueous solutions, such as for example 5 mM Tris/HCl, pH 8.5.

Further subject matter of the present invention relates to a kit for isolating RNA from a whole blood sample, containing at least the following components:

a) an aqueous lysis solution containing at least one lysis substance at a concentration of 1 mol/1 to 5 mol/l,

b) at least one detergent,

c) a proteinase, in particular proteinase K,

d) optionally, method instructions relating to the implementation of the method according to the invention, and

e) optionally, a solid phase for the attachment of the RNA.

The present invention relates, moreover, to the use of the kit according to the invention for isolating RNA from a whole blood sample.

### BRIEF DESCRIPTION OF THE SEVERAL VIEWS OF THE DRAWINGS

FIG. 1 is a graph showing a comparison of Cp values in connection with Example 1 described below.

FIG. 2 is a graph showing a comparison of RNA yield in connection with Example 1 described below.

FIG. 3 is a graph showing a comparison of Cp values in connection with Example 1 described below.

FIGS. 4A and 4B are graphs showing the quality of the isolated RNA, measured on the ratio A260/280, with FIG. 4A representing the isolation of RNA from whole blood samples by direct lysis and FIG. 4B representing selective erythrocyte lysis.

FIG. 5 is a graph showing RIN values from 5 different blood samples.

FIG. 6 is a graph reported in connection with Example 2 for eight blood samples.

FIG. 7 is a graph depicting RNA yield in connection with Example 4.

FIG. 8 is a graph summarizing the results of the investigation of the effect of the concentration of chaotropic salt upon the RNA yield.

FIG. 9 is a graph showing RNA purity dependency upon the chaotropic salt concentration.

FIG. 10 is a graph showing RNA quality dependency upon chaotropic salt concentration

FIG. 11 is a graph showing the effect of magnesium chloride on RNA yield.

FIG. 12 is a graph showing the effect of various bivalent ions on RNA yield.

FIG. 13 is a graph showing the effect of different detergents and detergent combinations upon the RNA yield and the lysis efficiency.

FIG. 14 is a graph showing the effect of different chaotropic salts upon RNA yield.

### DETAILED DESCRIPTION OF THE PREFERRED EMBODIMENT(S)

In the following the present invention is described in more detail using a number of exemplary embodiments.

### Example 1

### Lysis of Whole Blood Samples and Isolation of the RNA in the Minispin Format with Silica Membranes

For the lysis of the blood samples 200 μL whole blood are displaced with 200 μL lysis buffer and mixed strongly. A composition of the RNA blood lysis buffer according to the invention is as follows:

3 M GuSCN,

5% Brij 58 (w/v),

0.015% N-lauroyl-sarcosine,

100 mM MgCl₂.

Next proteinase K (20 mg/mL parent solution) is added, and it is incubated for 15 mins at RT. In the following this procedure according to the invention is called direct lysis.

Isolation of the Liberated RNA

In order to isolate the liberated RNA the conditions are set by adding 200 μL 70% ethanol (see step 9) such that the RNA can bind to a silica matrix. The further processing (from step 12 onwards) incl. DNA digestion is implemented with the commercially available RNA purification kit made by the company MACHEREY-NAGEL, NucleoSpin RNA II, REF 740955. This kit contains the following components:

Lysis buffer RA1 (not necessary here)
Wash buffer RA2
Wash buffer RA3 (concentrate)

### Membrane Desalting Buffer MDB

Reaction buffer for rDNase
rDNase, lyophilized
RNase-free water
NucleoSpin filter columns (not necessary here)
NucleoSpin RNA II columns
Collection tubes
User manual
Wash buffer RA2 contains GuSCN such as alcohol, wash buffer RA3 contains 80% ethanol, residual water and buffer substances.

As an alternative to the 200 μL sample, different quantities can also be processed. The ratios between the blood sample, the RNA blood lysis buffer and the ethanol are 1:1:1. With a volume of blood that is twice as high, the quantity of lysis buffer and ethanol is also doubled.

A schematic process sequence is shown in the following table:

| Step | |
|---|---|
| 1 | 200 μL whole blood in 1.5 mL tube |
| 2 | 200 μL RNA blood lysis buffer |
| 3 | Mix by inverting 3x |
| 4 | 20 μL proteinase K (approx. 20 mg/mL) |
| 5 | Mix (vortex) |
| 6 | Incubate for 15 mins at room temperature on the shaker: Eppendorf |
| | Thermoshake, 1400 rpm |
| 7 | Short centrifugation (1 s, 2000xg) |
| 8 | Add 200 μL 70% ethanol. |
| 9 | Shake strongly |
| 10 | Short centrifugation (1 s, 2000xg) |
| 11 | Reprocessing with the NucleoSpin RNA II kit. |
| | Pipette 700 μL of the lysate into a NucleoSpin RNA II column in |
| | the collection tube |
| 12 | Centrifuge for 30 s at 11000xg |
| 13 | Discard flow-through and collection tube |
| 14 | Insert the column into new collection tube |
| 15 | Add 350 μL MDB (Membrane Desalting Buffer) |
| 16 | Centrifuge for 1 min at 11000xg |
| 17 | Add 95 μL rDNase |
| 18 | Incubate for 15 mins at RT |
| 19 | Add 200 μL RA2 |
| 20 | Centrifuge for 30 s at 11000xg |
| 21 | Discard flow-through and collection tube |
| 22 | Insert the column into a new collection tube |
| 23 | Add 600 μL RA3 |
| 24 | Centrifuge for 30 s at 11000xg |
| 25 | Discard flow-through and insert the column into the collection tube |
| | again |
| 26 | Add 250 μL RA3 |
| 27 | Centrifuge for 2 mins at 11000xg |
| 28 | Insert the column into a nuclease-free collection tube (1.5 mL). |
| 29 | Add 60 μL RNase-free water |
| 30 | Centrifuge for 1 min at 11000xg |

The process sequence shows an example of the processing of 200 μL whole blood samples, but can be scaled up to any volumes. For this purpose the volumes of lysis buffer, proteinase and ethanol are increased by the same factor so that comparable lysis and binding conditions are obtained.

Analysis of the Isolated RNA

A whole series of investigation methods can be used for the assessment of the quantity and quality of RNA.

The RNA quantification is implemented by means of absorption measurements at 260 nm, and the quality is determined by means of quotients A260/280 and A260/228. This method is described in Short protocols in molecular biology. Ed. F. M. Ausubel. 1999 Wiley.

The essential feature for the quality of an isolated RNA is also the integrity of the latter. This is determined by means of Bioanalyzer measurements (Agilent 2100 Bioanalyzer) which are used by the RNA integrity number generated by the system as a measure of the RNA quality. The RNA Integrity Number, RIN, is a measure for the quality of the RNA and ranges from the ideal 10.0 to 0.

Furthermore, the suitability of the isolated RNA for the subsequent analysis is checked by means of RT PCR experiments. For this purpose a 73 nt RNA sequence is reverse-transcribed and amplified by means of PCR. The analysis takes place in the Roche LightCycler by means of real time PCR. If blood components, such as for example the haem, are displaced, PCR inhibition occurs.

Results:

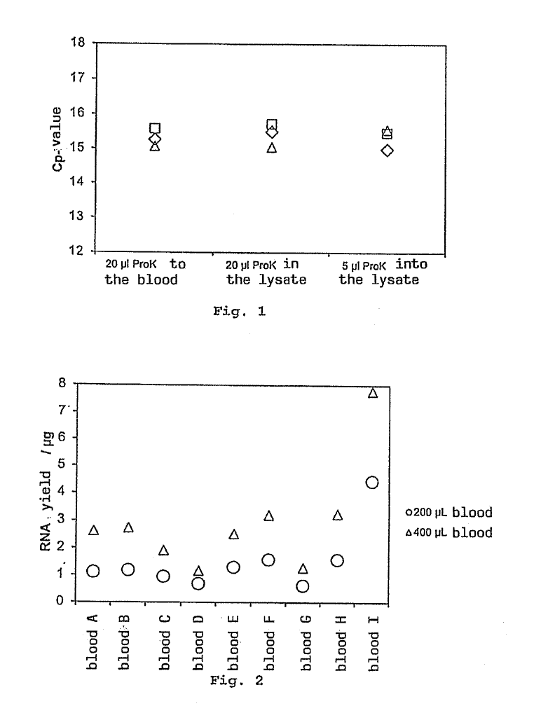

Whether in the sequence described above the proteinase K is added to the blood sample before adding the lysis buffer, or whether said blood sample is added to the blood/lysis buffer mixture has no effect upon the result. FIG. 1 shows a comparison of the two methods:

Approach 1 (left): Proteinase K was added directly to the blood sample.
Approach 2 (centre) and 3 (right): The blood sample was mixed with lysis buffer, then the proteinase was added (approach 2 with 20 μL proteinase K (20 mg/mL).
Approach 3: with 5 μL proteinase K (20 mg/mL)).

The individual values of 3 blood samples respectively (triangle, rhombus, square) are shown. The RNA quantity is shown here as a Cp value after the real time RT PCR. The results show, moreover, that the quantity of proteinase used is only of secondary significance. Even variation by the factor 4 provides comparable Cp values.

It is shown in FIG. 2 that the RNA yield increases as the quantity of blood is increased. The illustration shows the results of isolating RNA from whole blood samples by direct lysis using Minispin columns. A comparison is shown of the RNA yield with 200 and 400 μL whole blood. The quantity of RNA was determined by means of spectrophotometric determination at 260 nm. Blood A-I: fresh blood, individual donors, EDTA stabilised.

One can see the fluctuations to be expected between individual samples, and in all cases the yield when using a 400 μL sample is greater than with a 200 μL specimen. One can also see that factor 2 is evident in the RNA yield as well as in the sample volume. In the plurality of lysed samples in no case was there clogging of the columns, even in cases with very high haematocrit and correspondingly high RNA yields (see blood I).

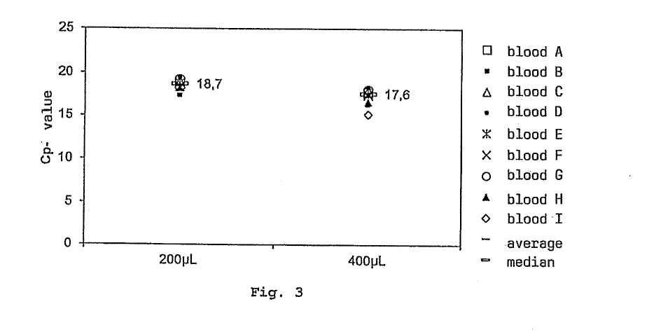

The difference in the yield when using 400 instead of 200 μL whole blood is also shown in the analysis with real time RT PCR. As shown by FIG. 3, the use of 400 μL blood leads to a Cp value reduction of approx. 1 cycle. Blood samples A-I are fresh blood from individual donors A-I, EDTA stabilised. Illustration of the individual values, as well as average and median.

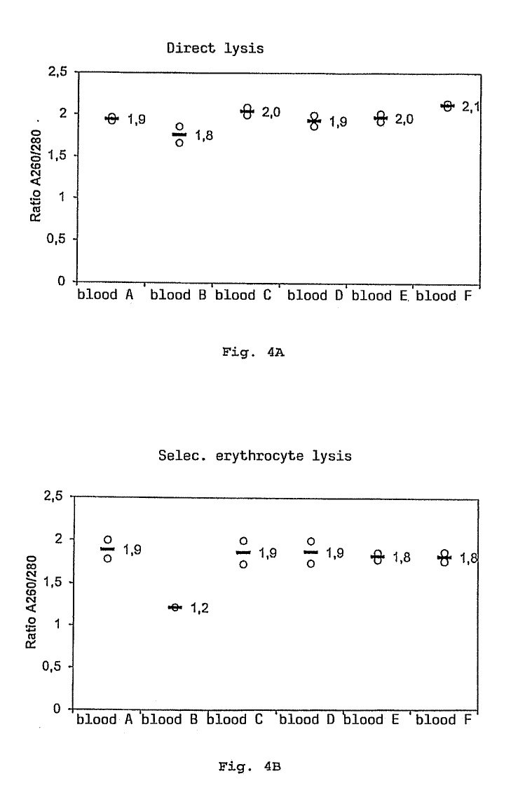

Due to the semi-logarithmic connection between the Cp value and RNA concentration, the decrease by one cycle corresponds to an increase in concentration by factor 2. This is the precise factor to be expected when doubling the quantity of blood. The quality of the isolated RNA, measured on the ratio A260/280, is shown in FIGS. 4A (isolation of RNA from whole blood samples by direct lysis) and 4B (selective erythrocyte lysis). The results are based on the processing of 400 μL whole blood respectively with direct lysis according to exemplary embodiment 1.

The selective erythrocyte lysis was implemented with the Qiagen kit, QIAamp RNA Blood Mini Kit, according to the attached manual. The A-F blood samples are once again fresh blood from individual donors A-F, EDTA stabilised. Illustration of the individual (n=2) and average values. Ideally RNA ratios A260/280 should have around 2.0-2.1. Generally, the quality of RNA isolated from blood should however be lower than that from other tissues or cells. The ratios determined by the method according to the invention are ideally between approximately 1.8 and 2.1.

For a comparison, FIG. 4B shows ratios which were obtained with a commercial kit based on selective erythrocyte lysis (Qiagen QIAamp RNA Blood Mini Kit). Here too the quality is good, however ratios over 1.9 are not achieved. Sample 2, which delivered a ratio of 1.8 with direct lysis, but of only 1.2 with selective erythrocyte lysis, is also striking. Due to the intricate and complex handling with the selective lysis, contamination of the RNA can not be ruled out.

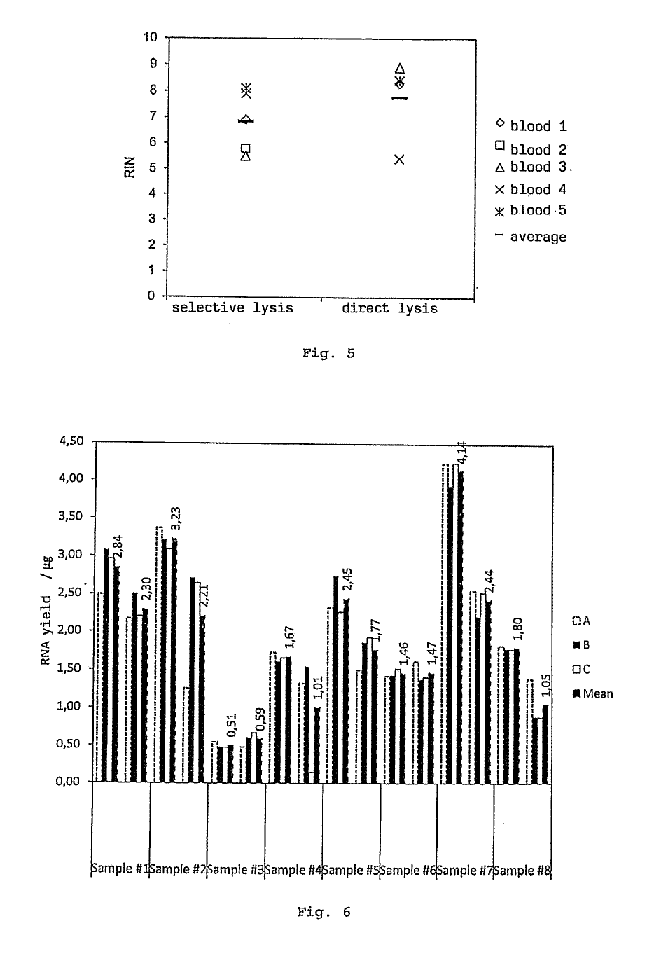

The RNA integrity that can be achieved with the method according to the invention is comparably high and comparable at the very least with standard methods which are based on the selective lysis of erythrocytes. In FIG. 5 RIN values from 5 different blood samples are shown. Here RNA was isolated from 400 μL whole blood samples by direct lysis (i.e. according to the invention) and selective erythrocyte lysis. Direct lysis: implementation according to Exemplary Embodiment 1. Selective erythrocyte lysis: implementation with Qiagen, QIAmp RNA Blood Mini Kit, according to the manual. Illustration of the RNA integrity (RIN values) after Bioanalyzer analysis. Blood 1-5: fresh blood, individual donors 1-5, EDTA stabilised. Illustration of the individual and average values.

The values of on average 7.8 achieved with the direct lysis are excellent, show high integrity of the RNA and prove in an impressive way that RNases are efficiently inactivated during the lysis and the cell disruption. For the comparison the RIN values are shown after selective erythrocyte lysis. With an average of 6.8 these come below the values of the direct lysis.

### Example 2

### Automation of the Method for Isolating RNA from Whole Blood—Processing in a Vacuum and in the Centrifuge

In the following a process sequence for processing up to 96 samples is shown. The lysis of the blood samples and the RNA isolation take place in 96-well plates. Here too the RNA is isolated after lysis with a commercially available RNA purification kit made by the company MACHEREY-NAGEL, NucleoSpin 8 RNA (REF 740698) or NucleoSpin 96 RNA (740709). These kits contain the following components:

Lysis buffer RA1 (not necessary here)
Wash buffer RA2
Wash buffer RA3 (concentrate)
Wash buffer RA4 (concentrate)
DNase reaction buffer
rDNase, lyophilized
RNase-free water
NucleoSpin RNA binding strips (with REF 740698) or
NucleoSpin RNA binding plate (with REF740709)
Wash plate
Square well block
Elution plate

Wash buffer RA2 contains GuSCN and alcohol, wash buffer RA3 (as above) and RA4 70% ethanol, residual water.

| Step | |
|---|---|
| 1 | Present 400 μL whole blood |
| 2 | Add 400 μL RNA blood lysis buffer |
| 3 | Mix (shaker or pipette on and off) |
| 4 | Add 10 μL proteinase K (approx. 20 mg/mL) |
| 5 | Mix (shaker, 5 s) |
| 6 | Incubate for 15 mins at room temperature, preferably on shaker |
| 7 | Add 400 μL 70% ethanol. |
| 8 | Mix (shaker or pipette on and off) |
| 9 | Apply the lysate to the NucleoSpin RNA binding plates or 48- |
| | well strips |
| 10 | From this point follow the NucleoSpin 8/96 RNA protocol. The |
| | processing can either take place in a centrifuge or in a vacuum. |

The extraction in the 96-well format is shown, for example, in FIG. 6. Here 8 blood samples respectively identified in triplicate were processed by centrifugation and in a vacuum. The quantity of RNA was determined spectrophotometrically at 260 nm. Specimens 1-8: fresh blood 400 μL, individual donors 1-8, n=3 (A, B, C), EDTA stabilised. Illustration of the individual and average values.

These results show that both possibilities for processing samples are possible. The fluctuations between the individual blood samples represents again the fluctuations in haematocrit (different quantities of leucocytes and so different RNA yields).

### Example 3

### Automation of the Method for Isolating RNA from Whole Blood—Processing Using Magnetic Beads

Silica membranes different from those described above can also be used as the solid phase for binding the isolated RNA. In the following exemplary embodiment magnetic beads are used instead of the membranes. The separation and isolation of the RNA does not take place here by centrifugation or a vacuum, but by magnetic separation on an appropriate separator, e.g. with static magnets such as the MACHEREY-NAGEL NucleoMag SEP, REF 744900 for 96-well plates.

In the following a process sequence for the processing of up to 96 samples is shown. The lysis of the blood samples and the RNA isolation take place in 96-well plates. Here the RNA is isolated after the lysis with a commercially available RNA purification kit made by the company MACHEREY-NAGEL, NucleoMag 96 RNA (REF 744350). The kit contains the following components:

Lysis buffer MR1 (not used here)
Binding buffer MR2
Wash buffer MR3
Wash buffer MR4
Elution buffer MR5
Magnetic beads
RNase free water
Reaction buffer for rDNase
rDNase (lyophilized)
Reducing agent TCEP (not used here).

The binding buffer MR2 contains >90% isopropanol, residual water. Wash buffer MR3 contains GuSCN as well as alcohol, wash buffer MR4, approx. 80% ethanol, residual water.

| Step | |
|---|---|
| 1 | Present 400 μL whole blood |
| 2 | Add 400 μL RNA blood lysis buffer |
| 3 | Mix (shaker or pipette on and off) |
| 4 | Add 10 μL proteinase K (approx. 20 mg/mL) |
| 5 | Mix (shaker, 5 s) |
| 6 | Incubate for 15 mins at room temperature, preferably on shaker |
| 7 | Add 28 μL magnetic beads and 400 μL binding buffer MR2. |
| 8 | Mix (shaker or pipette on and off) |
| 9 | From this point follow the NucleoMag RNA protocol. |

With this method too a plurality of blood samples were extracted. When using 400 μL whole blood 1-2 μg RNA with a ratio A260/280 of approximately 1.6 were isolated depending on the sample. The method according to the invention is therefore also suitable in combination with magnetic separation of RNA.

### Example 4

### RNA Extraction from Fresh and Frozen Human Whole Blood

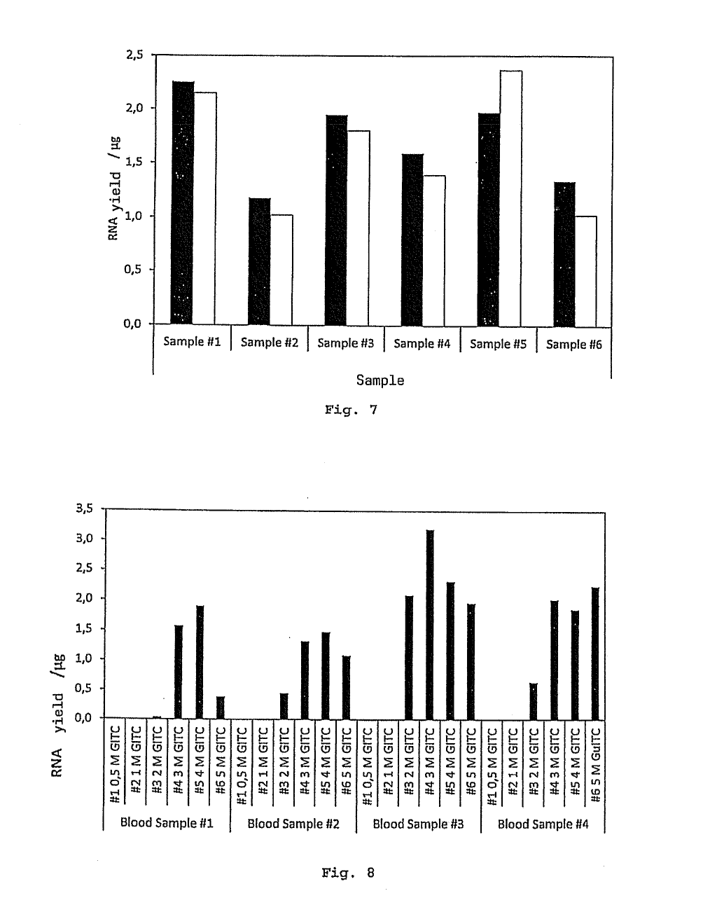

In this experiment the method according to the invention was implemented with frozen blood samples. For this purpose 6 fresh EDTA whole blood samples were divided up: Some of the samples were frozen at −20° C., and the others were stored at 4° for a period of 8 hrs as a comparison. Next the frozen samples were unfrozen, and in a parallel extraction the RNA was isolated according to Exemplary Embodiment 2. The RNA yield is shown in FIG. 7. The processing took place in a vacuum according to Exemplary Embodiment 2, and the quantity of RNA was determined spectrophotometrically at 260 nm. Black: fresh blood samples, white: frozen blood samples. Samples 1-6: fresh blood 200 μL, individual donors 1-6, EDTA stabilised. Presentation of the average values of n=3.

The results prove that the method according to the invention can be applied similarly both to fresh and to frozen samples. The RNA purity (A260/280), the RNA quality (RIN) and the amplifiability were also investigated by means of RT PCR. Here too there were no significant differences between the fresh and the frozen samples: the RNA purity and quality were comparable and no differences were observed either as regards amplifiability.

### Example 5

### Isolation of RNA from Animal Blood

As an example of the use of animal blood, at this point RNA was isolated from frozen horse blood according to Exemplary Embodiment 2. In each case, RNA could be isolated from a total of 5 individual blood samples. In no case was there any clogging of the binding plate or any other conspicuous features, and all of the samples could be processed in a vacuum without any problem. It could thus be shown that the method according to the invention is suitable not only for human whole blood, but also for blood samples from other species.

### Example 6

### Lysis Buffer Concentrations and Components

The lysis buffer according to the invention must guarantee that a number of objects are achieved after adding the blood sample. The four following pre-requisites must therefore be met:

1. The proteinase may not be inactivated by the buffer. It must be guaranteed that efficient protein digestion also takes place under the chosen high salt conditions.

2. RNases must be inactivated so as not to have any negative effect upon the quality of the isolated RNA.

3. The binding of the RNA to a solid phase must be possible in interaction with ethanol under the chosen conditions.

4. The lysis of the whole blood must be guaranteed.

Within this context the concentration of the chaotropic salt is particularly relevant. On the one hand the salt leads to lysis of the blood cells (feature 4), it inactivates the RNases (feature 2) and it leads to binding of the RNA to the solid phase (feature 3). However, if the concentration is too high, proteinases are inactivated and the lysis is incomplete; clogging of the column can not be prevented under these conditions (feature 1).

The conditions must therefore be chosen such that optimal interaction between the chaotropic salt and the proteinase is achieved. In a first experiment the GuSCN concentration in the lysis buffer according to Exemplary Embodiment 2 varies within the concentration range of 0.5, 1, 2, 3, 4 and 5 M. 4 individual blood samples were processed.

As a result it was established that the wells clogged with 0.5 and 1 M GuSCN. Despite the presence of proteinase in the preparation, the lysis efficiency of the lysis buffer (final GuSCN concentration 0.25 and 0.5 M) is not sufficient in order to fully digest the blood samples. Therefore, proteinase on its own does not lead to satisfactory lysis of the blood samples.

Next, the binding of RNA to the solid phase was investigated. The RNA yield is used here as the parameter. In FIG. 8 the results of the investigation of the effect of the concentration of chaotropic salt upon the RNA yield are summarised. The isolation of RNA was implemented by direct lysis from whole blood samples in the 96-well format with NucleoSpin 96 RNA. The processing was implemented in a vacuum and the quantity of RNA was determined spectrophotometrically at 260 nm. 4 individual blood samples were used. Presentation of the average values of n=3.

It can first of all be seen here that the GuSCN concentrations of 0.5 and 1 M did not deliver any yields because the wells clogged and isolation could not be implemented. A concentration of 2 M GuSCN already leads to the isolation of RNA, the method optimally being executed at concentrations of from 3 M, the RNA yields being higher here than with 2 M GuSCN.

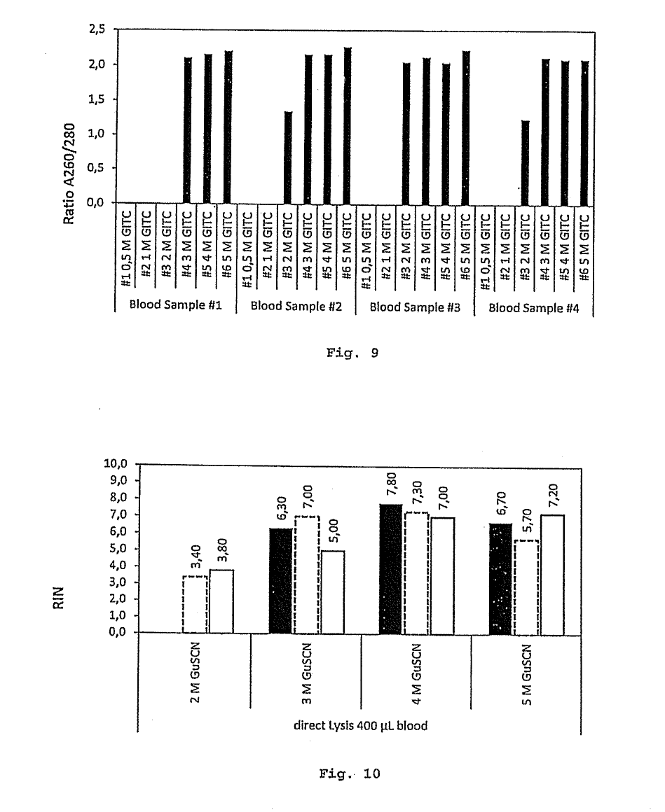

The RNA purity dependent upon the chaotropic salt concentration is shown in FIG. 9. The isolation of RNA from whole blood samples was implemented by direct lysis in the 96-well format with NucleoSpin 96 RNA. The processing was implemented in a vacuum, and the quantity of RNA was determined by spectrophotometric determination of the A260/280 ratio. 4 individual blood samples were used. Presentation of the average values of n=3.

Here too optimal results with ratios of approx. 2.1 were determined for GuSN concentrations of from 2 M. The method basically also functions with 1.5 M GuSCN, but the ratios are somewhat lower and the fluctuations over the blood samples are also somewhat greater.

The RNA quality dependent upon the chaotropic salt concentration is shown in FIG. 10.

The isolation of RNA from whole blood samples was implemented by direct lysis in the 96-well format with NucleoSpin 96 RNA. The processing was implemented in a vacuum and the RNA integrity (RIN) was determined by means of Bioanalyzer measurements. Samples from 3 individual blood samples (identified by the colours black, grey and white) were analysed. Sample 1 (black) with 2 M GuSCN clogged when the Bioanalyzer was running and did not provide any RIN. Presentation of the individual values.

High RIN values, and so high RNA quality and integrity were obtained for GuSCN concentrations >2 M. The method basically also functions with 1.5 or 2 M GuSCN, but the RIN values are somewhat lower than with higher concentrations, nevertheless RNases are clearly also at least partially inactivated here by the chaotropic salt. If the molarity of the lysis buffer is 3 M or higher, the inactivation is even more efficient, and this is made evident by increased RNA integrity and increased RIN values.

The effect of the chaotropic salt concentration upon the activity of the proteinase was examined in a separate experiment. The previously shown experiments according to Exemplary Embodiment 2 were basically repeated, but in the presence and in the absence of proteinase K. The results are summarised in the following table:

A master blood pool was used as a consistent sample for all of the approaches. The average values of n=3 are shown.

| Conc. | Without proteinase | | | | | | With proteinase | | | | | |
|---|---|---|---|---|---|---|---|---|---|---|---|---|
| GuSCN | 0.5 | 1 | 2 | 3 | 4 | 5 | 0.5 | 1 | 2 | 3 | 4 | 5 |
| Clogging | Yes | Yes | Yes | Yes | Yes | No | Yes | Yes | No | No | No | No |
| RNA yield | —/— | —/— | —/— | —/— | —/— | 0.39 | —/— | —/— | 1.2 | 2.6 | 2.6 | 2.6 |
| μg | | | | | | | | | | | | |
| RNA purity | —/— | —/— | —/— | —/— | —/— | 1.4 | —/— | —/— | 1.82 | 1.97 | 1.97 | 2.0 |
| A260/280 | | | | | | | | | | | | |
| RIN | —/— | —/— | —/— | —/— | —/— | —/— | —/— | —/— | 3.3 | 6.8 | 6.9 | 7.4 |

It is clear from this table that there is incomplete lysis if proteinase is not added, and the binding plate clogs. Only with chaotropic salt concentrations of 5 M was the lysis efficient such that no clogging was observed. The RNA yield and purity were low however.

Moreover, it was observed that the silica membrane was still clearly dark in colour after lysis and washing (contamination of cell residues and proteins), whereas after lysis with proteinase K the membranes were white and free from residues.

In the presence of proteinase chaotropic salt concentrations of >1 M lead to complete lysis (no clogging of the membrane). The RNA yield increases from 2 to 3 M, but then remains relatively constant. The same behaviour is seen with the RNA purity. This is good with 2 M, and better values are achieved as from 3 M GuSCN. The RNA quality, measured on the RIN, is likewise acceptable with 2 M GuSCN, and very good as from 3 M.

It can be noted in conclusion that even GuSCN concentrations of 4 and 5 M guarantee sufficiently high proteinase activity. If the proteinase is missing, the lysis is inefficient and incomplete.

### Example 7

### Effect of MgCl₂ and Other Bivalent Metal Ions Upon the Lysis Efficiency and the RNA Yield

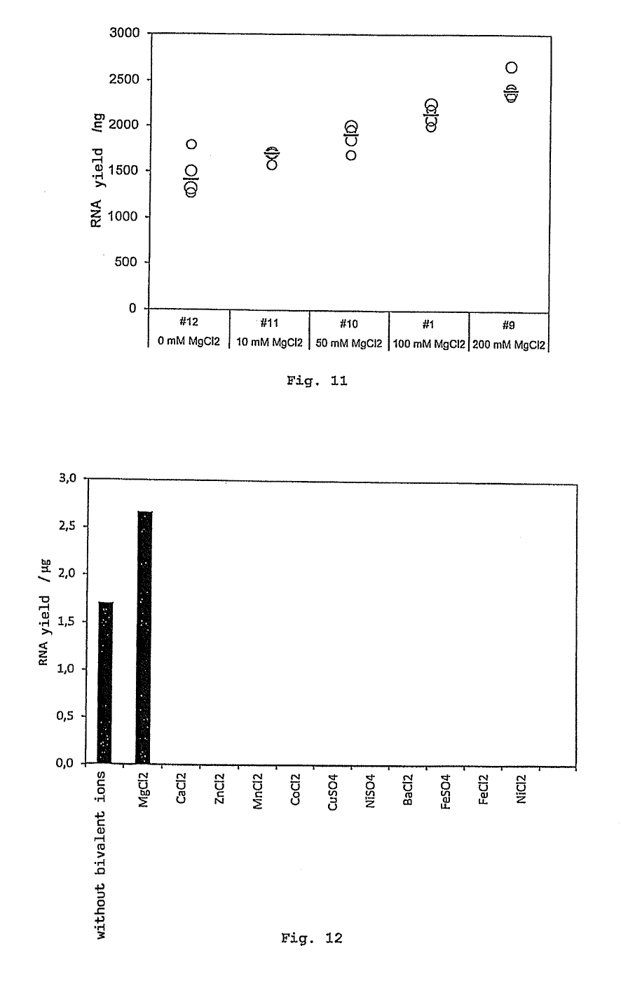

In a first experiment the concentration of the magnesium chloride in the lysis buffer was varied. This was carried out according to Exemplary Embodiment 1, and the results are summarised in FIG. 11. The RNA was isolated from whole blood by direct lysis according to Exemplary Embodiment 1. The RNA yield was determined by RiboGreen quantification. A blood pool was used, respectively n=4. Presentation of the individual values and medians.

The results demonstrate that by adding MgCl₂ a further increase in the RNA yield is possible, and this can be almost doubled by adding up to 200 mM MgCl₂ in the lysis buffer. Independently of the concentration of the magnesium ions the samples could be processed without any problem, and clogging of the membrane was not observed. In a further experiment according to Exemplary Embodiment 2 the quantity of MgCl₂ was increased even further. The following results were achieved here:

| Quantity of MgCl2 used | RNA yield |
|---|---|
| 0 mM | 1.22 μg |
| 100 mM | 2.28 μg |
| 200 mM | 2.36 μg |
| 400 mM | 2.24 μg |
| 800 mM | 2.04 μg |

This demonstrates that an increasing quantity of magnesium ions leads to an increase in the RNA yield. In the present case the optimum is approx. 200 mM; if the concentration increases further, the method according to the invention works in this way, but the RNA yield decreases slightly again with respect to the optimum.

This effect caused by the use of MgCl₂ is also surprising in so far as other bivalent ions (with the exception of calcium under certain circumstances) in the same concentration range lead to total clogging of the membrane. The ions cause precipitation, and so the samples can not be processed any further. The RNA yields are correspondingly low or zero. The RNA yield dependently upon the ions used is shown in FIG. 12. The RNA was isolated from whole blood by direct lysis according to Exemplary Embodiment 2. All of the metal ions were used at a concentration of 100 mM in the lysis buffer. The RNA yield was determined by RiboGreen quantification. A blood pool was used, respectively n=3, presentation of the average values. With the exception of the approaches without bivalent ions and with MgCl2, all other combinations led to clogging of the membrane, and so zero RNA yields.

In further experiments it was established that as well as magnesium ions, calcium ions also have a positive effect upon the RNA yield. When using calcium ions this effect is, however, additionally dependent upon the chaotropic salt used. It was thus observed that calcium ions (as CaCl₂) in combination with guanidinium thiocyanate have no boosting effect and clog the membranes. In combination with alkali thiocyanate such as sodium thiocyanate improvement of the RNA yield could, however, be observed. The calcium concentration with which this advantageous effect is particularly pronounced is approximately 50-100 mM.

These experiments show that the method according to the invention preferably works with magnesium ions, but also with calcium ions, and an improvement of the RNA yield is thereby achieved. However, other bivalent metal ions do not have a positive effect, but on the contrary a markedly negative effect because they lead to the precipitation of cell components.

### Example 8

### Use of Different Detergents in the Lysis Buffer

In the following the effect of detergents upon the lysis efficiency and RNA yield were examined. Detergents were used here individually or in a mixture. The following combinations were tested:

| Sample number | Non-ionic detergent | Anionic detergent |
|---|---|---|
| 1 | 50 g/L Brij58 | 0.15 g/L N-lauroyl- |
| | | sarcosine |
| 2 | 50 g/L Brij58 | — |
| 3 | 50 g/L Brij58 | 0.5 g/L SDS |
| 4 | 50 g/L Brij58 | 0.5 g/L docusate |
| 5 | 50 g/L Tween 20 | 0.15 g/L N-lauroyl- |
| | | sarcosine |
| 6 | 50 g/L Brij56 | 0.15 g/L N-lauroyl- |
| | | sarcosine |
| 7 | 50 g/L Brij35 | 0.15 g/L N-lauroyl- |
| | | sarcosine |
| 8 | 50 g/L Brij56 | 0.5 g/L SDS |

The chaotropic salt and the concentration of MgCl₂ was kept constant over all of the approaches (3 M GuSCN, 100 mM MgCl₂; implementation according to Exemplary Embodiment 2).

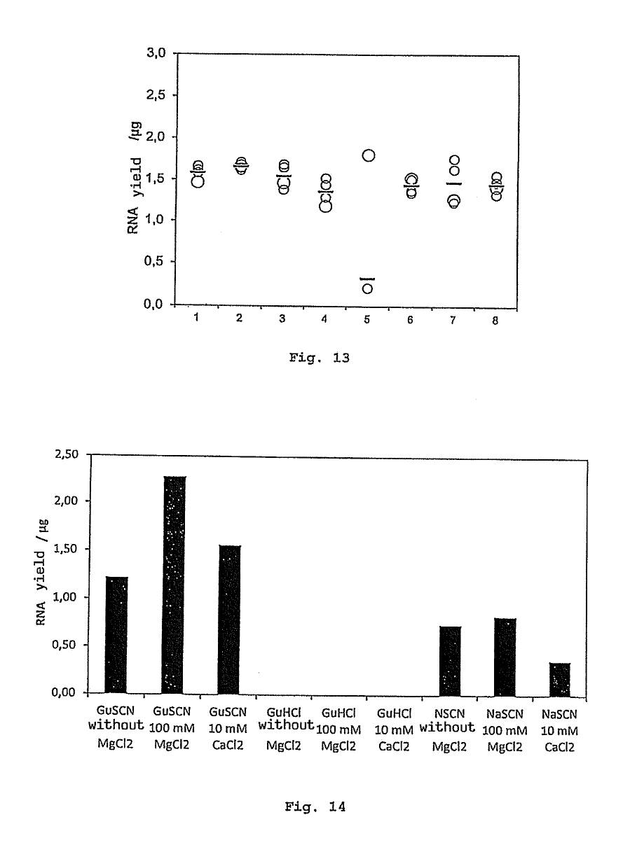

FIG. 13 shows the effect of different detergents and detergent combinations upon the RNA yield and the lysis efficiency. Isolation of RNA from whole blood by direct lysis according to Exemplary Embodiment 2. Individual values and median from n=4.

In conclusion it can be noted that both individual detergents and combinations of non-ionic and anionic detergents according to the method according to the invention can be used. Only combination No. 5 with Tween 20 led to greater yield fluctuations and on average to reduced RNA yields. Nevertheless, clogging of the membranes was not observed, and so the method according to the invention also works with Tween 20.

The detergent supports the lysis, releases lipids of the lysed blood cells and in this way prevents clogging of the membranes. If however one dispenses totally with the detergent, incomplete lysis and clogging of the membranes occurs.

### Example 9

### Use of Different Chaotropic Salts in the Lysis Buffer

In order to investigate the effect of different chaotropic salts, guanidinium thiocyanate (GuSCN), guanidine hydrochloride (GuHCl) and sodium thiocyanate (NaSCN) were tested as chaotropic substances in the lysis buffer according to Exemplary Embodiment 2, the chaotropic salt respectively being used at a concentration of 3 M. Average values of n=3. The results are shown in FIG. 14.

For guanidinium thiocyanate one can see the clear effect of magnesium ions upon the RNA yield, and likewise the boosting effect of calcium ions. When using sodium thiocyanate the RNA yield is generally lower, nevertheless the method works without bivalent ions, with 10 mM CaCl₂ and with 100 mM MgCl₂. When using guanidine hydrochloride the membranes clogged.

It can therefore be concluded that chaotropic salts based on thiocyanate ions, preferably guanidinium thiocyanate, are suitable for the method according to the invention.

The present invention has been described herein in terms of one or more preferred embodiments. However, it should be understood that numerous modifications and variations to these embodiments would be apparent to those skilled in the art upon a reading of the foregoing description. Therefore, it is intended that any such modifications and variations comprise a part of this invention, provided they come within the scope of the following claims and their equivalents.

1. A method for isolating RNA from a whole blood sample comprising the steps:
a) bringing the sample into contact with an aqueous lysis solution containing at least one lysis substance at a concentration of 1.5 mol/L to 7 mol/L and at least one detergent,
b) simultaneous or subsequent addition of a proteinase, in particular proteinase K, wherein preferably in the proteinase is added as a solid together with a solvent or as a proteinase solution, the respective quantity of liquid being set such that the quantity of liquid is at most 20% of the volume comprising the sample and the aqueous lysis solution, in particular at most 10%,
c) then incubating the solution obtained according to step b) for at least partial enzymatic digestion and lysis of the sample, a lysate being obtained, wherein in particular RNases contained in the solution in step c) are as far as possible totally inactivated, and
d) isolation of the RNA from the lysate.

2. (canceled)

3. The method according to claim 1, characterised in that the aqueous lysis solution contains the lysis substance at a concentration of 2 mol/L to 6 mol/L, in particular of 2.5 mol/L to 5 mol/L, preferably of 3 mol/L to 4 mol/L.

4. The method according to claim 1, characterised in that the aqueous lysis solution is mixed with the sample at a volume ratio of 1.8:1 to 1:1.8, in particular of 1.5:1 to 1:1.5, preferably of 1.2:1 to 1:1.2.

5. (canceled)

6. The method according to claim 1, characterised in that the sample is brought directly in contact with the lysis solution in step a) and is not diluted by adding a further solvent until the completion of step c), no stabiliser is added and/or it is not centrifuged, in particular the overall volume comprising the sample, the aqueous lysis solution and the proteinase exceeding the initial volume of the sample by no more than the factor 2.5, preferably by no more than the factor 1.5, by the completion of step c).

7. The method according to claim 1, characterised in that the lysis substance comprises one or more chaotropic salts, and in particular is selected from thiocyanates such as sodium thiocyanate, sodium thiocyanate and guanidinium thiocyanate, urea, perchlorate salts such as sodium perchlorate and/or potassium iodide and/or that the lysis substance includes potassium and/or sodium thiocyanate and the lysis contains calcium ions, the calcium ion concentration being in particular 10 to 150 mmol/L, preferably 50 to 100 mmol/L.

8. The method according to claim 1, characterised in that the lysis solution contains magnesium ions, in particular at a concentration of 10 to 1000 mmol/L, preferably of 100 to 600 mmol/L, more preferably 150 to 400 mmol/L and/or that the lysis solution is free from bivalent or multivalent metal ions with the exception of magnesium and calcium.

9. The method according to claim 1, characterised in that the lysis substance includes potassium and/or sodium thiocyanate and the lysis solution contains calcium ions, the calcium ion concentration being in particular 10 to 150 mmol/L, preferably 50 to 100 mmol/L.

10. The method according to claim 1, characterised in that the lysis solution is free from bivalent or multivalent metal ions with the exception of magnesium and calcium.

11. The method according to claim 1, characterised in that the detergent is selected from non-ionic detergents, anionic detergents or mixtures of the latter, in particular polyoxyethylene-20-cetyl ether (Brij 58®) or N-lauroyl-sarcosine.

12. The method according to claim 1, characterised in that the lysis solution has further ingredients which are chosen in particular from buffer substances, enzymes, reduction agents and/or alcohols.

13. The method according to claim 1, characterised in that in order to isolate the RNA from the lysate the RNA is attached to a solid phase, desirably followed by one or more washing steps.

14. The method according to claim 13, characterised in that for attachment of the RNA to the solid phase a binding agent is added to the lysate, in particular an alcohol, preferably ethanol and/or isopropanol, and an alcohol/water mixture.

15. The method according to claim 1, characterised in that the DNA is at least partially, preferably almost totally removed, in particular by DNase digestion.

16. The method according to claim 1, characterised in that the isolated RNA is extracted, in particular by desorption.

17. A kit for isolating RNA from a whole blood sample, containing at least the components:
a) an aqueous lysis solution containing at least one lysis substance at a concentration of 1 mol/L to 5 mol/L,
b) at least one detergent,
c) a proteinase, in particular proteinase K,
d) optionally, method instructions relating to the implementation of a method according to any of claims 1 to 16, and
e) optionally, a solid phase for the attachment of the RNA.

18. The use of a kit according to claim 17 for isolating RNA from a whole blood sample.

19. The method according to claim 3, characterised in that the aqueous lysis solution contains the lysis substance at a concentration of 2 mol/l to 6 mol/l, in particular of 2.5 mol/l to 5 mol/l, preferably of 3 mol/l to 4 mol/l.

20. The method according to claim 3, characterised in that the aqueous lysis solution is mixed with the sample at a volume ratio of 1.8:1 to 1:1.8, in particular of 1.5:1 to 1:1.5, preferably of 1.2:1 to 1:1.2.

21. The method according to claim 3, characterised in that the sample is brought directly in contact with the lysis solution in step a) and is not diluted by adding a further solvent until the completion of step c), no stabiliser is added and/or it is not centrifuged, in particular the overall volume comprising the sample, the aqueous lysis solution and the proteinase exceeding the initial volume of the sample by no more than the factor 2.5, preferably by no more than the factor 1.5, by the completion of step c).

22. The method according to claim 4, characterised in that the sample is brought directly in contact with the lysis solution in step a) and is not diluted by adding a further solvent until the completion of step c), no stabiliser is added and/or it is not centrifuged, in particular the overall volume comprising the sample, the aqueous lysis solution and the proteinase exceeding the initial volume of the sample by no more than the factor 2.5, preferably by no more than the factor 1.5, by the completion of step c).
